(19) United States
(12) Patent Application Publication
(10) Pub. No.: US 2022/0273619 A1
(43) Pub. Date: Sep. 1, 2022

(54) PHARMACEUTICAL FORMULATIONS COMPRISING SODIUM PALMITOYL-L-PROLYL-L-PROLYLGLYCYL-L-TYROSINATE AND METHODS FOR PREPARING THE SAME

(71) Applicant: Bridge Blctherapeutics, lnc., Gyeonggi-do (KR)

(72) Inventor: Sang Uk Kang, Gyeonggi-do (KR)

(73) Assignee: Bridge Blctherapeutics, lnc., Gyeonggi-do (KR)

(21) Appl. No.: 17/637,633

(22) PCT Filed: Aug. 3, 2020
(86) PCT No.: PCT/KR2020/010236

§ 371 Date: Feb. 23, 2022

(30) Foreign Application Priority Data
Aug. 23, 2019 (KR) 10-2019-0103882

(51) Int. Cl. A61K 31/4025; A61K 31/166; A61P 29/00

(52) U.S. Cl. CPC A61K 31/4025; A61K 31/166; A61P 29/00; A61K 38/07

(57) ABSTRACT

The present invention relates to a pharmaceutical formulation having excellent bioavailability and stability, comprising sodium palmitoyl-L-prolyl-L-prolyl-glycyl-L-tyrosinate (Compound I) as an active ingredient. The pharmaceutical formulation according to the present invention can be usefully used as a dosage form for treating inflammatory bowel disease and the like since Compound I, an active ingredient, is not decomposed in the stomach and released in the intestine.

### TECHNICAL FIELD

The present invention relates to a pharmaceutical formulation having excellent bioavailability and stability, comprising sodium palmitoyl-L-prolyl-L-prolyl-glycyl-L-tyrosinate. The pharmaceutical formulation according to the present invention can be used to prepare a pharmaceutical dosage form in which an active ingredient is not decomposed in the stomach and stably released in the intestine.

### BACKGROUND ART

Inflammatory bowel disease is largely divided into ulcerative colitis and Crohn's disease, and the cause of these is not yet known. Specifically, colitis is an inflammatory disease confined to the mucous membrane or submucosal layer of the large intestine. Inflammation or ulcer occurs from the rectum near the anus, and gradually progresses to the entire large intestine, and hemafecia, femafecia, diarrhea and abdominal pain occur. In severe cases, systemic symptoms such as fever, weight loss, and anemia appear. In some cases, it progresses acutely, but most often it occurs slowly from several weeks to several months. In addition, Crohn's disease is a disease in which lesions such as ulcers occur discontinuously in any site of the digestive tract from the mouth to the anus. In addition to abdominal pain, diarrhea, and hemafecia, in severe cases, symptoms such as fever, bloody discharge, weight loss, general malaise, and anemia appear.

Drugs that are currently used as therapeutic agents for inflammatory bowel disease are used for alleviating symptoms rather than direct treatment, and mainly include immunosuppressants, aminosalicylic acid formulation, adrenal cortical steroids and the like, but have been reported to have various side effects.

For example, infliximab, an immunosuppressant, has an effect by binding to TNF-α, and is used in the treatment of ulcerative colitis and Crohn's disease, but these treatments are expensive and cause severe allergic reaction (rash, itching, edema and the like) and various side effects such as chest pain in some patients.

In addition, 5-aminosalicylic acid (5-ASA) such as sulfasalazine, which blocks the production of prostaglandins, is classified as a drug having the least side effects among therapeutic agents for ulcerative colitis, but side effects still exist. For example, it is known that sulfasalazine causes side effects such as dyspepsia, headache, drug-induced connective tissue disorder, interstitial nephritis, anemia, reversible male infertility, nausea, vomiting, rash, liver disease, and leukopenia.

If the effect is not sufficient in the administration of 5-ASA, adrenal cortical steroids are administered in the short term. However, in active ulcerative colitis, the administration of steroid for less than 3 weeks indicates a risk of early relapse, and the initial treatment with prednisolone in amount of less than 15 mg/day is not effective. Steroids are effective in inducing remission, but can cause side effects in about 50% of patients and cause acne, mood swings, edema and the like. Furthermore, since side effects such as infectious disease, secondary adrenocortical insufficiency, peptic ulcer, diabetes mellitus, steroid kidney disease and the like may occur, there are limitations that should be used only in acute cases. Therefore, there is a need to develop a therapeutic agent that has an excellent effect of treating inflammatory bowel disease and does not cause side effects.

Palmitoyl-L-prolyl-L-prolyl-glycyl-L-tyrosine, the compound of Formula II below, has an excellent effect of inhibiting the expression of interleukin-6 and the activity of NF-κB (US2017/0008924), and it has been demonstrated to have excellent safety in various toxicity tests and is believed to have the effect of treating ulcerative colitis and Crohn's disease.

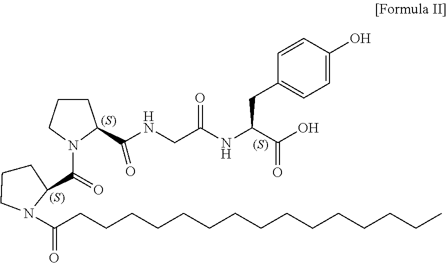

In order for palmitoyl-L-prolyl-L-prolyl-glycyl-L-tyrosine to effectively exhibit the effect of treating inflammatory bowel disease, the compound may be orally administered, and after reaching the large intestine without decomposition in the stomach, it is desirable to stay in the large intestine for some time. However, palmitoyl-L-prolyl-L-prolyl-glycyl-L-tyrosine has low water solubility and does not exhibit sufficient efficacy when administered in vivo, and in particular, there is a problem that the compound consisting of palmitic acid and natural amino acid present in nature is vulnerable to gastric acid and various digestive enzymes. In order to overcome this problem, the present inventors have studied for a long time the new salt form of palmitoyl-L-prolyl-L-prolyl-glycyl-L-tyrosine and a dosage form capable of being effectively delivered to the large intestine when administered orally. Based on the above, the present inventors completed the present invention.

### PRIOR ART DOCUMENT

### Patent Document

U.S. Patent Application Publication US 2017/0008924 (Jan. 12, 2017)

### DETAILED DESCRIPTION OF THE INVENTION

### Technical Problem

Inflammatory bowel disease (ulcerative colitis, Crohn's disease and the like) is an inflammatory disease confined to the mucous membrane or submucosal layer of the large intestine. It is desirable that the therapeutic agents for inflammatory bowel disease are not decomposed in the stomach when administered orally, and stay in the large intestine for some time after reaching the large intestine. In addition, drugs administered orally need to have sufficient water solubility for the dissolution in the gastrointestinal tract and absorption in the body.

Therefore, in order to allow palmitoyl-L-prolyl-L-prolyl-glycyl-L-tyrosine, which has low water solubility and is vulnerable to gastric acid and various digestive enzymes, to be able to express sufficient efficacy, it is an object of the present invention to provide a pharmaceutical formulation having excellent bioavailability and storage stability, comprising a new salt of palmitoyl-L-prolyl-L-prolyl-glycyl-L-tyrosine as an active ingredient.

### Solution to Problem

The present invention provides a pharmaceutical formulation for oral administration comprising sodium palmitoyl-L-prolyl-L-prolyl-glycyl-L-tyrosinate (Compound I), a compound of Formula 1 below, as an active ingredient:

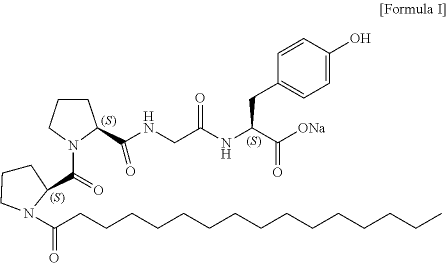

The pharmaceutical formulation may be tablets such as plain tablets, coated tablets, multi-layered tablets, or pressure-coated tablets, powders, granules, or capsule, and may be preferably tablets or capsules.

The pharmaceutical tablet of the present invention comprises an enteric polymer, and the enteric polymer may be at least one selected from the group consisting of a methacrylic acid-methyl methacrylate copolymer, a methyl acrylate-methyl methacrylate-methacrylic acid copolymer, a methacrylic acid-ethyl acrylate copolymer, cellulose acetate phthalate, hydroxypropyl methylcellulose phthalate, hydroxypropyl methylcellulose acetate succinate, polyvinyl acetate phthalate, cellulose acetate trimellitate, carboxymethyl ethyl cellulose and shellac, but is not limited thereto.

The enteric polymer may be a methacrylic acid-methyl methacrylate copolymer (Eudragit® L or Eudragit® S), a methyl acrylate-methyl methacrylate-methacrylic acid copolymer (Eudragit® FS 30D), or a mixture thereof. In addition, the enteric polymer may be a methacrylic acid-methyl methacrylate 1:1 copolymer (Eudragit® L100), a methacrylic acid-methyl methacrylate 1:2 copolymer (Eudragit® S100), or a mixture thereof, and the mixture may comprise a methacrylic acid-methyl methacrylate 1:1 copolymer and a methacrylic acid-methyl methacrylate 1:2 copolymer in a weight ratio of 1:1.

The pharmaceutical formulation of the present invention may comprise the enteric polymer in an amount of 5 to 500 parts by weight, 10 to 300 parts by weight, or 15 to 100 parts by weight based on 100 parts by weight of sodium palmitoyl-L-prolyl-L-prolyl-glycyl-L-tyrosinate, and may preferably comprise the enteric polymer in an amount of 20 to 80 parts by weight.

In addition, the pharmaceutical formulation of the present invention may further comprise at least one additive selected from the group consisting of microcrystalline cellulose, mannitol, hydroxypropyl methylcellulose (HPMC), polyethylene oxide, sodium croscarmellose, crospovidone, polyoxyglyceride, magnesium aluminometasilicate, and sodium starch glycolate, and may further comprise at least one additive selected from the group consisting of magnesium stearate, sodium starch glycolate, talc, and triethyl citrate (TEC).

When the pharmaceutical formulation of the present invention is administered, sodium palmitoyl-L-prolyl-L-prolyl-glycyl-L-tyrosinate may be released at a pH of 5.5 or higher, 6 or higher, 7 or higher, or 7.4 or higher. In addition, when the dissolution test is carried out for the pharmaceutical formulation of the present invention in a condition of 37° C. and 100 rpm according to the United States Pharmacopeia (USP) type 2 paddle method, 20% or less, 15% or less, 10% or less, or 5% or less of sodium palmitoyl-L-prolyl-L-prolyl-glycyl-L-tyrosinate may be dissolved in a pH 6.0 buffer for 1 hour, 2 hours, or 4 hours, and 80% or more, 85% or more, 90% or more, or 95% or more of sodium palmitoyl-L-prolyl-L-prolyl-glycyl-L-tyrosinate may be dissolved in a pH 7.4 buffer for 1 hour, 2 hours, or 4 hours.

In addition, the present invention provides a pharmaceutical formulation for oral administration for treating inflammatory bowel disease, comprising sodium palmitoyl-L-prolyl-L-prolyl-glycyl-L-tyrosinate (Compound I).

### Effect of the Invention

The pharmaceutical formulation of the present invention comprises Compound I having high water solubility as an active ingredient, and can delay the dissolution of Compound I until it reaches a non-acidic environment in which Compound I can be dissolved upon oral administration. In addition, since there is substantially no generation of impurities or no change in the dissolution pattern even in long-term storage, it can be usefully used in a pharmaceutical formulation for treating inflammatory bowel disease and the like that develops in the lower small intestine or the large intestine.

### BRIEF DESCRIPTION OF DRAWINGS

FIG. 1 is a typical scanning electron microscopy (SEM) image of sodium palmitoyl-L-prolyl-L-prolyl-glycyl-L-tyrosinate (Compound I).

### DETAILED DESCRIPTION FOR WORKING THE INVENTION

The pharmaceutical formulation of the present invention comprises sodium palmitoyl-L-prolyl-L-prolyl-glycyl-L-tyrosinate (Compound I), a compound of Formula 1 below, as an active ingredient:

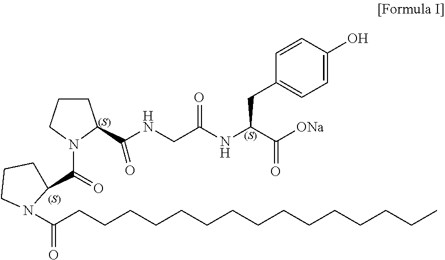

The pharmaceutical formulation may be tablets such as plain tablets, coated tablets, multi-layered tablets, or pressure-coated tablets, powders, granules, or capsule, and may be suitably tablets or capsules, and may comprise a pharmaceutically acceptable additive.

Pharmaceutically acceptable additives are those known in the art as natural or synthetic materials that are suitable for use in humans and animals since they do not have excessive side effects (such as, toxicity, irritation, or allergic reaction). As pharmaceutically acceptable additives, for example, diluents, binders, disintegrating agents, lubricants, stabilizing agents, coloring agents, flavors, surfactants and the like may be used.

As a diluent, starch, microcrystalline cellulose, lactose, glucose, mannitol, alginate, alkaline earth metal salt, clay, polyethylene glycol and dicalcium phosphate and the like may be used, but are not limited thereto.

As a binder, starch, microcrystalline cellulose, highly dispersible silica, mannitol, lactose, polyethylene glycol, polyvinylpyrrolidone, hydroxypropyl methylcellulose, hydroxypropyl cellulose, natural gum, synthetic gum, copovidone and gelatin and the like may be used, but are not limited thereto.

As a disintegrating agent, starch or modified starch such as sodium starch glycolate, corn starch, potato starch or pregelatinized starch, microcrystalline cellulose, low-substituted hydroxypropyl cellulose or alginic acid, crosslinked celluloses such as sodium croscarmellose, gums such as guar gum and xanthan gum, and crosslinked polymers such as crospovidone and the like may be used.

As a lubricant, talc, magnesium stearate, lauryl sulfate, hydrogenated vegetable oil, sodium benzoate, sodium stearyl fumarate, glyceryl monostearate and polyethylene glycol and the like may be used, and as a stabilizing agent, ascorbic acid, citric acid, butylated hydroxy anisole, butylhydroxy toluene, tocopherol derivatives and the like may be used.

Surfactants include sodium lauryl sulfate and poloxamer, a poly(oxyethylene-oxypropylene) block copolymer, as well as pharmaceutically acceptable additives such as polyoxyglyceride, magnesium aluminometasilicate, triethyl citrate (TEC) may be selected and used.

In the examples of the present invention, (silicified) microcrystalline cellulose, crospovidone, hydroxypropyl methylcellulose, magnesium stearate, talc, TEC and the like are used as such a additive, but the scope of the present invention is not limited to using the additive, and the above described additive may be included in a conventionally used dose by the selection of those of skill in the art.

The pharmaceutical formulation of the present invention may comprise an enteric polymer. The enteric polymer is able to allow Compound I, which is vulnerable to gastric acid and various digestive enzymes, to reach stably the large intestine and exhibit a therapeutic effect on inflammatory bowel disease and the like.

An enteric polymer having pH-dependent solubility in an aqueous environment of the gastrointestinal tract is known in the art and includes a methacrylic acid-methyl methacrylate copolymer, a methyl acrylate-methyl methacrylate-methacrylic acid copolymer, a methacrylic acid-ethyl acrylate copolymer, cellulose acetate phthalate, hydroxypropyl methylcellulose phthalate, hydroxypropyl methylcellulose acetate succinate, polyvinyl acetate phthalate, cellulose acetate trimellitate, carboxymethyl ethyl cellulose and shellac and the like.

A commercially available enteric polymer includes Eudragit® sold by Evonik Industries, and Eudragit® includes Eudragit® L 100 (a methacrylic acid-methyl methacrylate copolymer 1:1) and Eudragit® S 100 (a methacrylic acid-methyl methacrylate copolymer 1:2). Specifically, Eudragit® L 30 D-55 (a dispersion of a methacrylic acid-ethyl acrylate copolymer 1:1) and Eudragit® L-100-55 (a methacrylic acid-ethyl acrylate copolymer 1:1) have been reported to be dissolved at pH 5.5 or higher, and Eudragit® L100 (a methacrylic acid-methyl methacrylate copolymer 1:1) and Eudragit® L 12.5 (a solution of a methacrylic acid-methyl methacrylate copolymer 1:1) have been reported to be dissolved at pH 6.0 to 7.0, and Eudragit® S 100 (a methacrylic acid-methyl methacrylate copolymer 1:2), Eudragit® S 12.5 (a dispersion of a methacrylic acid-methyl methacrylate copolymer 1:2) and Eudragit® FS 30D (a dispersion of aqueous solution of a methyl acrylate-methyl methacrylate-methacrylic acid copolymer) have been reported to be dissolved at pH 7.0 or higher.

The pharmaceutical formulation of the present invention may further comprise anti-tacking agents and/or plasticizers, for example talc, triethyl citrate (TEC), glyceryl monostearate, acetyl triethyl citrate, acetyl tributyl citrate, polyethylene glycol, acetylated monoglyceride, glycerin, triacetin, propylene glycol, phthalate ester (for example, diethyl phthalate, dibutyl phthalate), titanium dioxide, ferric oxide, and the like.

In one embodiment, the pharmaceutical formulation of the present invention may comprise a methacrylic acid-methyl methacrylate copolymer as an enteric polymer, and may preferably comprise a methacrylic acid-methyl methacrylate copolymer 1:1 (Eudragit® L100), a methacrylic acid-methyl methacrylate copolymer 1:2 (Eudragit® S 100), or a mixture thereof.

In one embodiment, the pharmaceutical formulation of the present invention may comprise a methyl acrylate-methyl methacrylate-methacrylic acid copolymer (Eudragit® FS 30D) as an enteric polymer, and may further comprise PlasACRYL™ T20, which serves as an anti-tacking agent, a plasticizer, and a stabilizing agent.

In one embodiment, the pharmaceutical formulation of the present invention may be a matrix tablet comprising an enteric polymer in a matrix along with an active ingredient (Compound I) and other pharmaceutically acceptable additives, or a tablet coated with an enteric polymer.

In one embodiment, the pharmaceutical formulation of the present invention may be a capsule filled with a mixture of an active ingredient, an enteric polymer, and other pharmaceutically acceptable additives into a capsule, wherein the active ingredient may be filled into a capsule in the form of granules coated with an enteric polymer. In addition, the enteric polymer may coat a capsule containing an active ingredient. The capsule may be a gelatin capsule or an HPMC capsule, but is not limited thereto.

The pharmaceutical formulation according to the present invention may be prepared by methods known in the art, for example dry or wet granulation, roller compression or direct compression process.

In addition, in the pharmaceutical formulation according to the present invention, a method for preparing a coating layer may be appropriately selected from conventional methods for preparing a coating layer by those of skill in the art, and includes a fluidized bed coating method, a pan coating method, a dry coating method, and the like. The coating layer may be formed using a coating agent, a coating aid, or a mixture thereof. Also, in addition to an enteric coating to which an enteric polymer is applied, a seal coating (for example, Opadry Clear or Opadry AMB, manufactured by Colorcon) may be further applied.

The pharmaceutical formulation of the present invention may be prepared by 1) a method of mixing and compressing an active ingredient with an enteric polymer to prepare a tablet, 2) a method of treating an active ingredient with an enteric polymer to prepare granules and then filling a capsule with the granules, or 3) a method of filling a capsule with an active ingredient, and then coating the capsule with an enteric polymer, and the like.

In the pharmaceutical formulation of the present invention, an enteric polymer may be included in an amount of 5 to 500 parts by weight, 10 to 300 parts by weight, or 15 to 100 parts by weight based on 100 parts by weight of an active ingredient, and may be preferably included in an amount of 20 to 80 parts by weight.

In one embodiment, the present invention provides a pharmaceutical formulation for oral administration for treating inflammatory bowel disease, comprising sodium palmitoyl-L-prolyl-L-prolyl-glycyl-L-tyrosinate (Compound I).

In one embodiment, in the pharmaceutical formulation of the present invention, the active ingredient is dissolved in a condition of pH 7 or higher.

In one embodiment, for the pharmaceutical formulation of the present invention, when the dissolution test in vitro is carried out at 37° C. and 100 rpm in a solvent of 500 mL of 0.1 N HCl for 1 to 2 hours, in a pH 6.0 buffer for 1 to 4 hours, in a pH 7.4 buffer for 1 to 6 hours according to the USP type 2 paddle method, the active ingredient is not substantially dissolved in a condition of 1 N HCl and pH 6.0, and 90% or more of the active ingredient is released at pH 7.4 within 4 hours.

In one embodiment, when the pharmaceutical formulation of the present invention is stored for 1 to 6 months in a long-term storage stability condition (25° C./60% RH) and an accelerated stability condition (40° C./75% RH), the content of the active ingredient remains substantially the same, and there is substantially no generation of new impurities or no increase in impurities, and the dissolution pattern before and after storage is substantially the same.

Therefore, the pharmaceutical formulation of the present invention can delay the dissolution of the active ingredient until it reaches a non-acidic environment in which the active ingredient (Compound I) can be rapidly dissolved, and thus the pharmaceutical formulation of the present invention can be very usefully applied to treat inflammatory bowel diseases and the like, which require the release of the active ingredient into lesions of the lower small intestine or the large intestine.

Hereinafter, the embodiments and examples of the present application will be described in detail with reference to the accompanying drawings so that a person of ordinary skill in the art to which the present invention belongs can easily practice. However, the present application can be implemented in various forms and is not limited to the embodiments and examples described herein.

Throughout the specification of the present application, unless otherwise stated, when a certain part “comprises” a certain component, it means that the part can further comprise other components, not exclude other components.

Throughout the specification of the present application, the term “about” means that the number or range is not limited to the exact number or range in which the number or range is presented, but that the number or range includes a value around the cited number or range as understood by those of skill in the art depending on the context in which the number or range is used.

### Preparation Example 1

### Preparation of Palmitoyl-L-prolyl-L-prolyl-glycyl-L-tyrosine (Compound II)

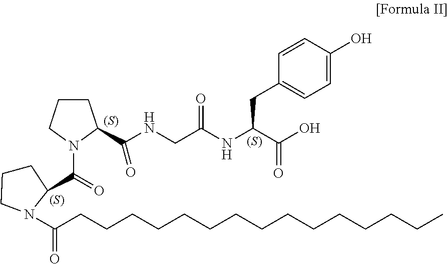

The compound of Formula II above, palmitoyl-L-prolyl-L-prolyl-glycyl-L-tyrosine, was prepared according to the method described in U.S. patent application Ser. No. 15/205,853, the content of which is incorporated herein by reference in its entirety.

### Preparation Example 2

### Preparation of Sodium Palmitoyl-L-prolyl-L-prolyl-glycyl-L-tyrosinate (Compound I)

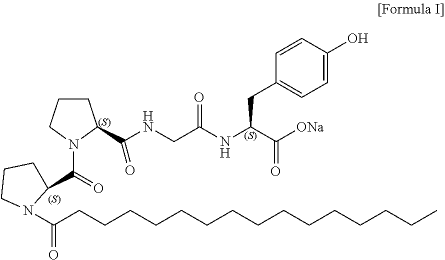

Palmitoyl-L-prolyl-L-prolyl-glycyl-L-tyrosine may be treated with a sodium base such as Na₂CO₃, NaHCO₃ or NaOH and converted to sodium palmitoyl-L-prolyl-L-prolyl-glycyl-L-tyrosinate (Compound I).

For example, 8.6 kg of NaHCO₃ was added to reactor 1, and 452 kg of water was added thereto. The NaHCO₃ aqueous solution in reactor 1 was transferred to another container A, and then 45 kg of palmitoyl-L-prolyl-L-prolyl-glycyl-L-tyrosine was added to reactor 1. 368 kg of the solution in the container A was added to reactor 1 and stirred for 1 to 3 hours at 20 to 30° C. 82 kg of the solution in the container A was added to reactor 1 and stirred for 1 to 5 hours at 20 to 30° C. The temperature was raised to 45-55° C., and then the mixture was further stirred for 3 to 5 hours. After the reaction was completed, the resulting sodium palmitoyl-L-prolyl-L-prolyl-glycyl-L-tyrosinate (Compound I) was centrifuged to remove water, and then washed with 66 kg of acetone. After drying, 44.2 kg of the resulting Compound I was placed in an LDPE bag and a fiber drum and stored for further treatment.

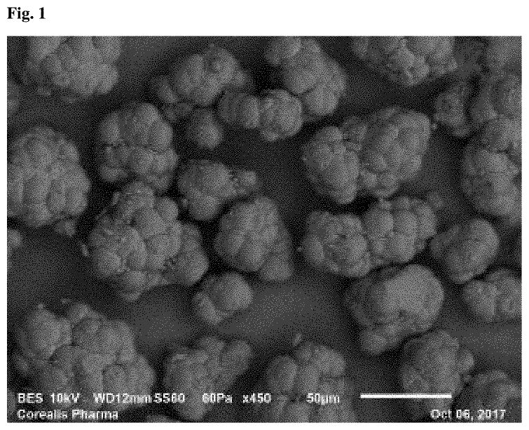

42.7 kg of 44.2 kg of Compound I was added to reactor 1, and 396 kg of tetrahydrofuran (THF) was added to reactor 1, and then heated to 40-50° C. to dissolve completely. The dissolved solution was filtered to remove impurities, and THF was removed under reduced pressure, and then 100 kg of THF was added again to dissolve completely at 40-50° C. 360 kg of acetone was further added and stirred at 40-50° C. for 1 to 2 hours. The temperature of the reactor was lowered to −5 to 5° C., and the obtained solid was dried under reduced pressure to obtain 37.48 kg of the final compound (sodium palmitoyl-L-prolyl-L-prolyl-glycyl-L-tyrosinate (Compound I)). The image of Compound I was confirmed by scanning electron microscopy (SEM). The confirmed image is shown in FIG. 1, and as shown in FIG. 1, Compound I exhibited a nearly circular shape.

¹H NMR (400 MHz, DMSO-d₆) δ 9.38 (brs, 1H), 8.13 (t, 1H, J=5.6 Hz), 7.25 (d, 1H, J=6.4 Hz), 6.87 (d, 2H, J=8.0 Hz), 6.55 (d, 2H, J=8.4 Hz), 4.49 (dd, 1H), 4.27 (dd, 1H), 3.90 (dd, 1H), 3.57-3.44 (m, 6H), 2.95-2.78 (m, 2H), 2.20 (m, 2H), 2.08-1.7 (m, 8H), 1.44 (m, 2H), 1.42 (s, 24H), 0.85 (t, 3H, J=6.4 Hz); LCMS (m/z) 671.5 (MH⁺ of free form, palmitoyl-L-prolyl-L-prolyl-glycyl-L-tyrosine).

### Preparation Example 3

### Preparation of Disodium Palmitoyl-L-prolyl-L-prolyl-glycyl-L-tyrosinate

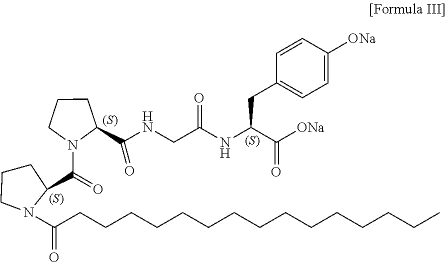

In the method for preparing Compound I, an excess of NaOH (for example; 4 equivalents) was added to prepare disodium palmitoyl-L-prolyl-L-prolyl-glycyl-L-tyrosinate. However, it was confirmed that since it was very hygroscopic, it could not be maintained in the form of the solid powder.

### Test Example 1

### Solubility of Palmitoyl-L-prolyl-L-prolyl-glycyl-L-tyrosine (Compound II)

The solubility was tested in various solvents for palmitoyl-L-prolyl-L-prolyl-glycyl-L-tyrosine (Compound II). The solubility test was performed by manual dilution combined with visual observation. The results of the experiment are shown in Table 1.

TABLE 1
Solubility Results at Room Temperature

| | Solubility | | Solubility |
|---|---|---|---|
| Solvent | (mg/mL) | Solvent | (mg/mL) |
| Methanol | 1-5 | Heptane | <1 |
| Ethanol | <1 | Cyclohexane | <1 |
| Isopropyl Alcohol | <1 | 1,4-dioxane | <1 |
| 1-Butanol | <1 | DMSO | 10-25 |
| Acetonitrile | <1 | DMF | 1-5 |
| Acetone | <1 | N-methyl pyrrolidone | 1-5 |
| Methyl Ethyl Ketone | <1 | Water | <1 |
| Methyl Isobutyl Ketone | <1 | Methanol-H₂O (1:1) | <1 |
| Ethyl Acetate | <1 | Methanol-H₂O (3:1) | <1 |
| Isopropyl Acetate | <1 | Ethanol-H₂O (1:1) | <1 |
| Methyl t-Butyl Ether | <1 | Ethanol-H₂O (3:1) | <1 |
| Tetrahydrofuran | <1 | ACN-H₂O (1:1) | <1 |
| 2-Methyl Tetrahydrofuran | <1 | Acetone-H₂O (1:2) | <1 |
| Toluene | <1 | THF-H₂O (1:1) | 1-5 |

As shown in Table 1 above, it was confirmed that palmitoyl-L-prolyl-L-prolyl-glycyl-L-tyrosine (Compound II) had low solubility in most organic solvents and water (<1 mg/mL).

### Test Example 2

### Measurement of Solubility of Micronized Palmitoyl-L-prolyl-L-prolyl-glycyl-L-tyrosine (Compound II)

In order to increase the solubility of palmitoyl-L-prolyl-L-prolyl-glycyl-L-tyrosine (Compound II) in water, Compound II was micronized to have a particle size of less than 5 μm, and its solubility was measured.

The solubility was measured by the following method. An appropriate amount of sample was placed in a 1.5 mL HPLC vial, and then 1.0 mL of solvent was added. The HPLC vial was shaken at 25° C. at a speed of 700 rpm, then the slurry was filtered, and the filtrate was analyzed by HPLC. In this case, the limit of quantification (LOQ) was 2 μg/mL.

The results of measuring the solubility are shown in Table 2 below.

TABLE 2

| Name | Solvent | Solubility (mg/mL) |
|---|---|---|
| Compound II | Water | <LOQ |
| | SGF (pH = 1.2) | <LOQ |
| | FaSSIF | 0.01 |
| Micronized Compound II | Water | <LOQ |
| (Particle Size <5 μm) | SGF (pH = 1.2) | <LOQ |
| | FaSSIF | 0.05 |

Limit of Quantification (LOQ) = 2 μg/mL.
SGF: Simulated Gastric Fluid
FaSSIF: Fasted State Simulated Intestinal Fluid

As shown in Table 2 above, it was confirmed that there was no significant difference in the solubility between Compound II and the micronized Compound II.

### Test Example 3

### Measurement of Solubility of Amorphous Solid Dispersion Form of Palmitoyl-L-prolyl-L-prolyl-glycyl-L-tyrosine (Compound II)

In order to increase the solubility of palmitoyl-L-prolyl-L-prolyl-glycyl-L-tyrosine (Compound II) in water, the amorphous solid dispersions of Compound II were prepared, and then the solubility was measured.

The measurement results are shown in Tables 3 and 4 below.

TABLE 3

| Name | | Media | Solubility (mg/mL) |
|---|---|---|---|
| Compound II: | Compound II: | Water | <LOQ |
| Kollidon | Kollidon VA64 | SGF (pH =1.2) | <LOQ |
| VA64 = 1:1 | 1:1 Simple Mixture | FaSSIF | 0.01 |
| | Amorphous Solid | Water | <LOQ |
| | Dispersion | SGF (pH = 1.2) | <LOQ |
| | | FaSSIF | 0.34 |
| Compound II: | Compound II: | Water | <LOQ |
| Kollidon | Kollidon VA64 | SGF (pH = 1.2) | <LOQ |
| VA64 = 1:2 | 1:2 Simple Mixture | FaSSIF | 0.01 |
| | Amorphous Solid | Water | <LOQ |
| | Dispersion | SGF (pH = 1.2) | <LOQ |
| | | FaSSIF | 0.06 |
| Compound II: | Compound II: | Water | <LOQ |
| PVP K30 = 1:1 | PVP K30 | SGF (pH = 1.2) | <LOQ |
| | 1:1 Simple Mixture | FaSSIF | 0.01 |
| | Amorphous Solid | Water | <LOQ |
| | Dispersion | SGF (pH = 1.2) | <LOQ |
| | | FaSSIF | 0.09 |
| Compound II: | Compound II: | Water | <LOQ |
| PVP K30 = 1:2 | PVP K30 | SGF (pH = 1.2) | <LOQ |
| | 1:2 Simple Mixture | FaSSIF | <LOQ |
| | Amorphous Solid | Water | <LOQ |
| | Dispersion | SGF (pH = 1.2) | <LOQ |
| | | FaSSIF | 0.12 |

Limit of Quantification (LOQ) = 2 μg/mL.

TABLE 4

| Name | | Media | Solubility (mg/mL) |
|---|---|---|---|
| Compound II: | Compound II: | Water | <LOQ |
| HPMC | HPMC E3 = | SGF (pH = 1.2) | <LOQ |
| E3 = 1:1 | 1:1 Simple Mixture | FaSSIF | 0.01 |
| | Amorphous Solid | Water | <LOQ |
| | Dispersion | SGF (pH = 1.2) | <LOQ |
| | | FaSSIF | 0.35 |
| Compound II: | Compound II: | Water | <LOQ |
| HPMC | HPMC E3 = | SGF (pH = 1.2) | <LOQ |
| E3 = 1:2 | 1:2 Simple Mixture | FaSSIF | 0.01 |
| | Amorphous Solid | Water | <LOQ |
| | Dispersion | SGF (pH = 1.2) | <LOQ |
| | | FaSSIF | 0.55 |
| Compound II: | Compound II: | Water | <LOQ |
| HPMC | HPMC ASMG = | SGF (pH = 1.2) | <LOQ |
| ASMG = 1:1 | 1:1 Simple Mixture | FaSSIF | <LOQ |
| | Amorphous Solid | Water | <LOQ |
| | Dispersion | SGF (pH = 1.2) | <LOQ |
| | | FaSSIF | 0.01 |
| Compound II: | Compound II: | Water | <LOQ |
| HPMC | HPMC ASMG = | SGF (pH = 1.2) | <LOQ |
| ASMG = 1:2 | 1:2 Simple Mixture | FaSSIF | <LOQ |
| | Amorphous Solid | Water | <LOQ |
| | Dispersion | SGF (pH = 1.2) | <LOQ |
| | | FaSSIF | <LOQ |

Limit of Quantification (LOQ) = 2 μg/mL.

As shown in Tables 3 and 4 above, it was confirmed that there was no significant difference in the solubility between the simple mixture and the solid dispersion.

### Test Example 4

### Measurement of Solubility of Palmitoyl-L-prolyl-L-prolyl-glycyl-L-tyrosine (Compound II) to which Surfactant is Added

In order to increase the solubility of palmitoyl-L-prolyl-L-prolyl-glycyl-L-tyrosine (Compound II) in water, a surfactant such as sodium lauryl sulfate (SLS) was added, and the solubility was measured. The results of measuring the solubility are shown in Table 5 below.

TABLE 5

| Name | | Media | Solubility (mg/mL) |
|---|---|---|---|
| Compound II | 1% SLS | Water | 0.47 |
| | | SGF (pH = 1.2) | 0.09 |
| | | FaSSIF | 1.99 |
| | 5% SLS | Water | 1.92 |
| | | SGF (pH = 1.2) | 0.38 |
| | | FaSSIF | 5.09 |
| | 1% Poloxamer 188 | Water | <LOQ |
| | | SGF (pH = 1.2) | <LOQ |
| | | FaSSIF | 0.01 |
| | 5% Poloxamer 188 | Water | <LOQ |
| | | SGF (pH = 1.2) | <LOQ |
| | | FaSSIF | 0.01 |

Limit of Quantification (LOQ) = 2 μg/mL.

As shown in Table 5 above, it was confirmed that there was no significant increase in the solubility when the surfactant was added.

### Test Example 5

### Solubility of Sodium Palmitoyl-L-prolyl-L-prolyl-glycyl-L-tyrosinate Salt (Compound I) and Other Salts

The solubility was tested at room temperature in various solvents for Compound I. In addition, the solubility test was performed by manual dilution combined with visual observation. Specifically, 2 mg of Compound I was added to a 1.5 mL HPLC vial and continuously stirred at ambient temperature while the solvent was gradually added. The results of measuring the solubility are shown in Table 6 below.

TABLE 6

| Solvent | Solubility (mg/mL) |
|---|---|
| Methanol | >100 |
| Ethanol | 50-100 |
| Isopropyl alcohol | 50-100 |
| 1-Butanol | 50-100 |
| Acetonitrile | <1 |
| Acetone | <1 |
| Methyl Ethyl Ketone | 3.3-5.0 |
| Methyl Isobutyl Ketone | 1.2-1.4 |
| Ethyl Acetate | <1 |
| Isopropyl Acetate | <1 |
| Methyl t-Butyl Ether | <1 |
| Tetrahydrofuran | >100 |
| 2-Methyl Tetrahydrofuran | >100 |
| Toluene | 50-100 |
| Heptane | <1 |
| Cyclohexane | <1 |
| 1,4-Dioxane | 33.3-50 |
| DMSO | 50-100 |
| DMF | 20-33.3 |
| N-Methyl pyrrolidone | 50-100 |
| Water | >100 |
| Methanol-H₂O (1:1) | >100 |
| Methanol-H₂O (3:1) | >100 |
| Ethanol-H₂O (1:1) | 50-100 |
| Ethanol-H₂O (3:1) | 50-100 |
| Acetonitrile-H₂O (1:1) | 50-100 |
| Acetone-H₂O (1:2) | 50-100 |
| Tetrahydrofuran-H₂O (1:1) | 50-100 |

As shown in Table 6 above, it was confirmed that the water solubility of Compound I was higher by tens of thousands of times or more compared to that of Compound II in an acid form. Specifically, it was confirmed that the solubility of Compound II was lower than the limit of quantification (LOQ, 2 μg/mL), but the solubility of Compound I was 100 mg/mL or more.

In addition to Compound I, various salts of the compound were prepared from Compound II, and the solubility in water was measured, and the results are shown in Table 7 below.

TABLE 7

| Salt Form of Compound II | Solubility (mg/mL) |
|---|---|
| Calcium Salt | <1 |
| Magnesium Salt | <1 |
| Zinc Salt | <1 |
| Meglumine Salt | <10 |
| Arginine Salt | <10 |

As shown in Table 7 above, the various salts of Compound II were prepared, but it was confirmed that they all exhibited a low solubility of 10 mg/mL or less in water. As a result, it can be seen that the sodium salt form (Compound I) of Compound II disclosed in U.S. Patent Application Publication US 2017/0008924 has the excellent pharmaceutical properties and is therefore most suitable for development as a medicament.

Hereinafter, the present invention will be described in more detail through the additional experiments on various dosage forms comprising Compound I as an active ingredient, but the following examples are provided only for the purpose of illustration and are not intended to limit the scope of the present invention.

Dissolution Test

The dissolution test was performed by the USP type II paddle method in a condition of 37° C., 100 rpm. Specifically, the dissolution tests were performed in an acidic environment by using 0.1 N HCl, in an acidic environment of pH 6.0 by adding a buffer solution (the fine adjustment of pH was performed by 5 N HCl), and in a neutral environment of pH 7.4, respectively.

The method for preparing the buffer solution and a 0.1 N HCl solution is as follows.

Preparation of a 0.1 N HCl solution: Based on the preparation of 24 L of a solution, 198 mL of hydrochloric acid was added to 24 L of purified water and mixed well.

Preparation of a buffer solution: Based on the preparation of 6 L of a solution, 255.44 g of sodium phosphate tribasic dodecahydrates (Na₃PO₄.12H₂O) was added to 6 L of purified water and mixed well. The concentration of this buffer solution is 112 mM.

Stability Test

After the dosage forms prepared in the present invention were stored for a period of time in a long-term storage stability condition (25° C./60% RH) or an accelerated stability condition (40° C./75% RH), the content of individual impurities or total impurities was measured. The impurities were measured using a validated HPLC analysis method. The specific conditions are as follows.

TABLE 8
Chromatography Condition

| Column | Zorbax SB-C8 (250 × 4.6 mm, 5 μm) | | |
|---|---|---|---|
| Column Temperature | 50° C. | | |
| Autosampler Temperature | Ambient (20° C.) | | |
| Injection Volume | 10 μl (Needle Wash: 50% Methanol) | | |
| Mobile Phase A | 0.2% Trifluoroacetic Acid in Water | | |
| Mobile Phase B | 0.2% Trifluoroacetic Acid in Acetonitrile | | |
| | | Mobile | Mobile |
| Gradient | Time (min) | Phase A (%) | Phase B (%) |
| | 0.0 | 90 | 10 |
| | 5.0 | 30 | 70 |
| | 20.0 | 5 | 95 |
| | 20.1 | 90 | 10 |
| | 30.0 | 90 | 10 |

| | |
|---|---|
| Flow rate | 1.0 mL/min |
| Detection Wavelength | 220 nm |
| Runtime | 30 minutes |

### Example 1

### Preparation of Enteric Coated Granules

Compound I was enteric coated with Eudragit L100/S100 (Eudragit L100:S100=1:1 (w/w)), talc, and triethyl citrate using anhydrous ethanol as a solvent. The specific composition of the granules is shown in Table 9 below.

TABLE 9

| Ingredient | Weight Ratio (%) |
|---|---|
| Compound I | 70 |
| Eudragit L100/S100 | 19 |
| Talc | 9 |
| Triethyl Citrate | 2 |
| Anhydrous Ethanol | (—)* |
| Sum | 100 |

*Removed during the process

For the enteric coated granules of Example 1, the dissolution test of Compound I was performed in an acidic environment of pH 6.0 and in a neutral environment of pH 7.4. The dissolution test results are shown in Table 10.

TABLE 10

| | Dissolution Rate of Example 1 (%) | |
|---|---|---|
| | Environment: 900 ml Buffer | Environment: 900 ml Buffer |
| | pH 6.0 | pH 7.4 |
| Time (hr) | Paddles: 100 rpm | Paddles: 100 rpm |
| 1 | 33 | 61 |
| 2 | 1 | 61 |
| 4 | 0 | 60 |

As shown in Table 10 above, the granules of Example 1 exhibited a dissolution rate of 33% after 1 hour in an acidic environment and maintained a dissolution rate of about 60% after 1 hour in a neutral environment. It was believed that the dissolution was not confirmed after 2 hours in an acidic environment, because Compound I was converted to Compound II (water solubility <2 μg/mL) in an acidic environment while the surface of the granules was gradually dissolved.

### Examples 2 and 3

Preparation of Granules to which Seal Coating and Enteric Coating are Applied

Compound I was seal coated with Opadry Clear or Opadry AMB using water as a solvent and then enteric coated with Eudragit FS 30D/Plasacryl T20. The specific composition of the granules is shown in Table 11 below.

TABLE 11

| | | Weight Ratio (%) | |
|---|---|---|---|
| Item | Ingredient | Example 2 | Example 3 |
| Active Ingredient | Compound I | 70 | |
| Seal Coating | Opadry Clear | 5 | — |
| | Opadry AMB | — | 5 |

| | | |
|---|---|---|
| | Water USP | (—)* |
| Enteric Coating | Eudragit FS 30 D | 22.5 |
| | Plasacryl T20 | 2.5 |
| | Water USP | (—)* |

| | |
|---|---|
| Sum | 100 |

*Removed during the process

For the granules of Examples 2 and 3, the dissolution test of Compound I was performed in an acidic environment of pH 6.0 and in a neutral environment of pH 7.4. The dissolution test results are shown in Table 12 below.

TABLE 12

| | Dissolution Rate of Example 2 (%) | | Dissolution Rate of Example 3 (%) | |
|---|---|---|---|---|
| | Environment: 900 | Environment: 900 | Environment: 900 | Environment: 900 |
| Time | ml Buffer pH 6.0 | ml Buffer pH 7.4 | ml Buffer pH 6.0 | ml Buffer pH 7.4 |
| (hr) | Paddles: 100 rpm | Paddles: 100 rpm | Paddles: 100 rpm | Paddles: 100 rpm |
| 1 | 59 | 65 | 65 | 69 |
| 2 | 70 | 65 | 62 | 69 |
| 4 | 66 | 65 | 1 | 69 |

As shown in Table 12 above, the granules of Examples 2 and 3 exhibited a dissolution rate of about 60% after 1 hour in an acidic environment. In addition, similar to Example 1, a phenomenon in which the detection amount of Compound I was partially decreased over time in an acidic environment was observed.

### Example 4

### Preparation of Direct Compression Tablet

200 mg of Compound I was co-milled with microcrystalline cellulose and crospovidone and then compressed with magnesium stearate to prepare the tablets. The specific composition of the tablets is shown in Table 13.

TABLE 13

| Ingredient | Weight Ratio (%) | mg/unit |
|---|---|---|
| Compound I | 50 | 200 |
| Microcrystalline Cellulose | 44 | 176 |
| Crospovidone | 5 | 20 |
| Magnesium Stearate | 1 | 4 |
| Sum | 100 | 400 |

### Example 5

### Preparation of Direct Compression Tablet

200 mg of Compound I was dry blended with Eudragit L100, silicified microcrystalline cellulose and magnesium stearate and then compressed to prepare the tablets. The specific composition of the tablets is shown in Table 14.

TABLE 14

| Ingredient | Weight Ratio (%) | mg/unit |
|---|---|---|
| Compound I | 40 | 200 |
| Eudragit L 100 | 30 | 150 |
| Silicified Microcrystalline Cellulose | 29 | 145 |
| Magnesium Stearate | 1 | 5 |
| Sum | 100 | 500 |

For the tablets of Example 5, the dissolution test of Compound I was performed in an acidic environment of pH 6.0 and in a neutral environment of pH 7.4. The dissolution test results are shown in Table 15 below.

TABLE 15

| | Dissolution Rate of Example 5 (%) | |
|---|---|---|
| | Environment: 900 ml Buffer | Environment: 900 ml Buffer |
| | pH 6.0 | pH 7.4 |
| Time (hr) | Paddles: 100 rpm | Paddles: 100 rpm |
| 1 | 1 | 55 |
| 2 | 0 | 69 |
| 4 | 0 | 95 |

As shown in Table 15 above, for the tablets of Example 5, it was confirmed that Compound I was not substantially dissolved in an acidic environment, and the dissolution of Compound I was gradually increased in a neutral environment, and the dissolution rate of Compound I was 95% after 4 hours.

### Examples 6 and 7

### Preparation of Direct Compression Tablet

25 mg or 200 mg of Compound I was dry blended with Eudragit S100, silicified microcrystalline cellulose and magnesium stearate and then compressed to prepare the tablets. The specific composition of the tablets is shown in Table 16.

TABLE 16

| | Weight Ratio | mg/unit | |
|---|---|---|---|
| Ingredient | (%) | Example 6 | Example 7 |
| Compound I | 40 | 25.0 | 200 |
| Eudragit S100 | 20 | 12.5 | 100 |
| Silicified Microcrystalline Cellulose | 39 | 24.4 | 195 |
| Magnesium Stearate | 1 | 0.6 | 5 |
| Sum | 100 | 62.5 | 500 |

After the tablets of Examples 6 and 7 were stored for 1 month in an accelerated stability (40° C./75% RH) condition, the amount of impurities produced and the dissolution rate (%) before and after storage were compared. The results of the experiment are shown in Table 17.

TABLE 17

| | | Example 6 | | Example 7 | |
|---|---|---|---|---|---|
| Accelerated Condition | | T = 0 | T = 1 month | T = 0 | T = 1 month |
| Storage Period | | | | | |
| (40° C./75% RH) | | | | | |
| Individual Impurities | | Not Detected | Not Detected | Not Detected | Not Detected |
| | | (<0.1%) | (<0.1%) | (<0.1%) | (<0.1%) |
| Total Impurities | | Not Detected | Not Detected | Not Detected | Not Detected |
| | | (<0.1%) | (<0.1%) | (<0.1%) | (<0.1%) |
| Dissolution | | | | | |
| Environment | Hour | | | | |
| Acidic Environment | 2 | All tablets were partially | | All tablets were partially | |
| (500 ml 0.1N HCl) | | disintegrated | | disintegrated | |
| After Dissolving | 1 | 60 | 58 | 47 | 48 |
| for 2 Hours in | 2 | 83 | 84 | 101 | 103 |
| Acidic Environment, | 4 | 99 | 96 | 105 | 103 |
| Neutral Environment | 6 | 96 | 95 | 105 | 103 |
| (Na₃PO₄, pH 7.4) | | | | | |

As shown in Table 17 above, it was confirmed that the impurities were not generated in both Examples 6 and 7. In addition, it was confirmed that all tablets were partially disintegrated in an acidic environment for 2 hours, and the dissolution progressed in a neutral environment.

It was confirmed that the dissolution rates before and after storage in an accelerated condition were substantially the same. Therefore, the tablets have excellent storage stability, and have a high dissolution rate of Compound I in an environment of the lower small intestine or the large intestine.

### Example 8

Preparation of Tablets to which Seal Coating and Enteric Coating are Applied

200 mg of Compound I was dry blended with the additives and then compressed to prepare the tablets. Seal coating was first performed in an aqueous solution using Opadry clear (HPMC/HPC) on the prepared tablets, and enteric coating was performed in an aqueous solution using Eudragit FS 30D and Plasacryl T20. The specific composition of the tablets is shown in Table 18.

TABLE 18

| | | Weight Ratio | |
|---|---|---|---|
| Item | Ingredient | (%) | mg/unit |
| Core | Compound I | 40 | 200 |
| | HPMC | 20 | 100 |
| | Mannitol | 20 | 100 |
| | Microcrystalline Cellulose | 15 | 75 |
| | Sodium Croscarmellose | 4 | 20 |
| | Magnesium Stearate | 1 | 5 |
| Core Sum | | 100 | 500 |
| Seal Coating | Opadry clear | — | 25 |
| | Water USP | — | (—)* |
| Enteric Coating | Eudragit FS 30 D | — | 119 |
| | Plasacryl T20 | — | 12 |
| | Water USP | — | (—)* |

| | |
|---|---|
| Total Weight of Tablet | 656 |

*Removed during the process

### Example 9

### Preparation of Capsules

200 mg of Compound I, polyethylene oxide, and crospovidone were mixed by V-blender for 3 minutes, co-milled, and then talc was added, and mixed by V-blender for 2 minutes again. The final mixture was filled into HPMC capsules. The specific composition of the capsules is shown in Table 19.

TABLE 19

| Ingredient | Weight Ratio (%) | mg/unit |
|---|---|---|
| Compound I | 50 | 200 |
| Polyethylene Oxide | 44 | 176 |
| Crospovidone | 5 | 20 |
| Talc | 1 | 4 |
| Sum | 100 | 400 |
| HPMC Capsule “0” | — | 96* |
| Capsule Weight | — | 496 |

*Average weight of 10 empty capsules

### Example 10

### Preparation of Capsules

Compound I (50%) and HPMC (50%) were dissolved using a methanol/dichloromethane mixed solvent. The solution was co-precipitated using a spray dryer and filled into HPMC capsules to complete enteric formulations. The specific composition of the capsules is shown in Tables 20 and 21 below.

TABLE 20

| Ingredient | Weight Ratio (%) |
|---|---|
| Compound I | 50 |
| HPMC | 50 |
| Methanol/Dichloromethane | (900) * |
| Mixed Solvent (1:1 w/w) | |
| Sum | 100 |

* Removed during the process

TABLE 21

| Ingredient | mg/unit |
|---|---|
| Product of Table 20 (Compound I:HPMC = 1:1) | 400 |
| HPMC Capsule “0” | 96* |
| Capsule Weight | 496 |

*Average weight of 10 empty capsules

### Example 11

### Preparation of Capsules

200 mg of Compound I, magnesium aluminometasilicate, polyoxyglyceride, and microcrystalline cellulose were wet granulated with anhydrous ethanol and co-milled. It was lubricated with sodium starch glycolate and magnesium stearate and then filled into HPMC capsules of size 0. The specific composition of the capsules is shown in Table 22.

TABLE 22

| Ingredient | Weight Ratio (%) | mg/unit |
|---|---|---|
| Compound I | 40 | 200 |
| Polyoxyglyceride | 21 | 104 |
| Magnesium Aluminometasilicate | 21 | 104 |
| Microcrystalline Cellulose | 15 | 75 |
| Sodium starch Glycolate | 3 | 13 |
| Magnesium Stearate | 1 | 4 |
| Anhydrous Ethanol | (35) * | (—)* |
| Sum | 100 | 500 |
| HPMC Capsule “0” | — | 96** |
| Capsule Weight | — | 596 |

*Removed during the process;
**Average weight of 10 empty capsules

### Example 12

Preparation of Capsules Filled with Enteric Coated Granules

Compound I was enteric coated with Eudragit FS 30D/Plasacryl T20 using a VFC Lab Micro fluid bed and using water as a solvent (Table 23). The enteric coated granules were mixed with magnesium stearate in a ratio of 99.5:0.5 (w/w) and filled into HPMC size 2 capsules to prepare the capsules. The specific composition of the capsules is shown in Tables 23 and 24.

TABLE 23

| Ingredient | Weight Ratio (%) |
|---|---|
| Compound I | 75 |
| Eudragit FS 30D | 22.5 |
| Plasacryl T20 | 2.5 |
| Water USP | (—)* |
| Sum | 100 |

*Removed during the process

TABLE 24

| Ingredient | Weight Ratio (%) | mg/capsule |
|---|---|---|
| Enteric Coated Granule of Table 23 | 99.5 | 268 |
| Magnesium Stearate | 0.5 | 1.3 |
| Sum | 100 | 269 |
| HPMC Capsule “2” | — | 61* |
| Capsule Weight | — | 330 |

*Average weight of 10 empty capsules

### Examples 13 and 14

Preparation of Capsules Filled with Enteric Coated Granules

25 mg or 200 mg of Compound I was enteric coated directly with Eudragit L100/S100 (Eudragit L100:S100=1:1 (w/w)), triethyl citrate (TEC), talc, and anhydrous ethanol. The enteric coated granules were filled into HPMC capsules. The specific composition of the capsules is shown in Table 25.

TABLE 25

| | | mg/unit | |
|---|---|---|---|
| Ingredient | Weight Ratio (%) | Example 13 | Example 14 |
| Compound I | 60 | 25 | 200 |
| Eudragit L100/S100 (1/1), TEC, Talc | 40 | 17 | 133 |
| Anhydrous Ethanol | — | (—)* | (—)* |
| Sum | 100 | 42 | 333 |
| HPMC Capsule | — | 48** | 75*** |

| | | |
|---|---|---|
| Capsule Weight | 90 | 408 |

*Removed during the process
**Average weight of 10 empty capsules “Size 3”;
***Average weight of 10 empty capsules “Size 1”

The capsules filled with the enteric coated granules (Example 13) were tested for the stability and dissolution. The results of the experiment are shown in Table 26 below.

TABLE 26
Accelerated Condition

| Storage Period | | Example 13 | | Example 14 | |
|---|---|---|---|---|---|
| (40° C./75% RH) | | T = 0 | T = 1 month | T = 0 | T = 1 month |
| Total Impurities (%) | | 0.55 | 0.62 | 0.51 | 0.61 |
| Dissolution | | | | | |
| Environment | Hour | | | | |
| Acidic Environment | 2 | All capsules | All capsules | All capsules | All capsules |
| (500 ml 0.1 N | | were partially | were partially | were | were swellen |
| HCl) | | disintegrated | disintegrated | | |
| After Dissolving | 1 | 105 | 100 | 80 | 84 |
| for 2 Hours in | 2 | 107 | 104 | 91 | 95 |
| Acidic Environment, | 4 | 107 | 104 | 104 | 102 |
| Neutral Environment | 6 | 107 | 103 | 105 | 104 |
| (Na₃PO₄, pH 7.4) | | | | | |

As shown in Table 26 above, it was confirmed that Compound I was stable for 1 month in an accelerated stability condition (40° C./75% RH). In addition, it was confirmed that there was almost no generation or increase of impurities.

As a result of the dissolution test, it was confirmed that when the enteric capsules were exposed to a 0.1 N HCl dissolution solution for 2 hours, all the capsules were partially disintegrated or swellen. In addition, it was confirmed that, in a dissolution solution adjusted to pH 7.4 using a sodium phosphate buffer (Na₃PO₄ buffer), Compound I was released within 1 hour in substantially all capsules.

Therefore, it can be seen that the capsule can delay the release of Compound I until it reaches a non-acidic environment in which Compound I can be rapidly released. This property can be very useful in the dosage form of therapeutic agents for inflammatory bowel diseases, which require the release of the active ingredient such as Compound I into lesions of the lower small intestine or the large intestine.

### Examples 15 and 16

Preparation of Capsules Filled with Enteric Coated Granules

25 mg or 200 mg of Compound I was enteric coated granulated by a high-shear granulation method. The granulation was performed at 50° C. using anhydrous ethanol, and the granules were dried in an oven and then filled into HPMC capsules. The specific composition of the capsules is shown in Table 27.

TABLE 27

| | | mg/unit | |
|---|---|---|---|
| Ingredient | Weight Ratio (%) | Example 15 | Example 16 |
| Compound I | 75 | 25 | 200 |
| Eudragit S 100 | 20 | 7 | 53 |
| HPMC | 4 | 1 | 11 |
| Anhydrous Ethanol | (28) * | (—)* | (—)* |
| Magnesium Stearate | 1 | 0.3 | 3 |
| Sum | 100 | 33 | 267 |
| HPMC Capsule | — | 48** | 75*** |

| | | |
|---|---|---|
| Capsule Weight | 81 | 342 |

*Removed during the process
**Average weight of 10 empty capsules “Size 3”;
***Average weight of 10 empty capsules “Size 1”

### Example 17

Preparation of Capsules to which Enteric Coating is Applied

200 mg of Compound I was first filled into HPMC size 2 capsules, and the capsules were enteric coated with Eudragit FS 30D and Plasacryl T20 in aqueous solution. The composition of the capsules according to the present method is shown in Table 28.

TABLE 28

| | Item | Ingredient | mg/unit |
|---|---|---|---|
| | Active Ingredient | Compound I | 200 |
| | Capsule | HPMC Capsule “2” | 61* |
| Core Sum | | | 261 |
| | Enteric Coating | Eudragit FS 30D | 59 |
| | | Plasacryl T20 | 6 |
| | | Water USP | (—)** |

| | |
|---|---|
| Total Sum | 326 |

*Average weight of 10 empty capsules;
**Removed during the process

For the granules of Example 17, the dissolution test of Compound I was performed in an acidic environment of pH 6.0 and in a neutral environment of pH 7.4. The results are shown in Table 29.

TABLE 29

| | Dissolution Rate of Example 17 (%) | |
|---|---|---|
| | Environment: 900 ml Buffer pH 6.0 | Environment: 900 ml Buffer pH 7.4 |
| Time (hr) | Paddles: 100 rpm | Paddles: 100 rpm |
| 1 | 0 | 0 |
| 2 | 0 | 91 |
| 4 | 0 | 96 |

As shown in Table 29, in the capsules of Example 17, the dissolution of Compound I did not substantially occur in an acidic environment, and the dissolution of Compound I was gradually increased in a neutral environment, resulting in a dissolution rate of Compound I of 96% after 4 hours.

### Examples 18 and 19

Preparation of Capsules to which Enteric Coating is Applied

25 mg or 200 mg of Compound I was mixed with magnesium stearate in a weight ratio of 99:1 (Compound I:magnesium stearate), and the mixture was filled into HPMC capsules and then enteric coated with Eudragit L100/S100 (Eudragit L100:S100=1:1 (w/w)), triethyl citrate (TEC), and talc using anhydrous ethanol in a fluid bed. The composition of the capsules according to the present method is shown in Table 30.

TABLE 30

| | | mg/unit | |
|---|---|---|---|
| Ingredient | Weight Ratio (%) | Example 18 | Example 19 |
| Compound I | 99 | 25 | 200 |
| Magnesium Stearate | 1 | 0.3 | 2.4 |
| HPMC Capsule | — | 48* | 48* |
| Core Sum | 100 | 73 | 250 |
| Eudragit L100/S100 | — | 70** | 70** |
| (1:1), TEC, Talc | | | |

| | | |
|---|---|---|
| Capsule Weight | 143 | 320 |

*Average weight of 10 empty capsules
**Solid ingredients consisting of 48 mg of Eudragit L/S100 and 22 mg of TEC/talc

The enteric coated capsules (Examples 18 and 19) were tested for the stability and dissolution. The results are shown in Table 31.

TABLE 31
Accelerated Condition

| Storage Period | | Example 18 | | Example 19 | |
|---|---|---|---|---|---|
| (40° C./75% RH) | | T = 0 | T = 1 month | T = 0 | T = 1 month |
| Total Impurities (%) | | 0.64 | 0.66 | 0.58 | 0.66 |
| Dissolution | | | | | |
| Environment | Hour | | | | |
| Acidic Environment | 2 | No change in capsule | | No change in capsule | |
| (500 ml 0.1 N HCl) | | | | | |
| After Dissolving | 1 | 97 | 97 | 101 | 72 |
| for 2 Hours in | 2 | 99 | 99 | 106 | 105 |
| Acidic Environment, | 4 | 99 | 99 | 106 | 105 |
| Neutral | 6 | 99 | 99 | 106 | 106 |
| Environment | | | | | |
| (Na₃PO₄, pH | | | | | |
| 7.4) | | | | | |

As shown in Table 31 above, it was confirmed that Compound I was stable for 1 month in an accelerated stability condition (40° C./75% RH), and there was almost no generation or increase of impurities.

As a result of the dissolution test, it was confirmed that when the enteric coated capsules were exposed to a 0.1 N HCl dissolution solution for 2 hours, all the capsules were stable. It was confirmed that, in a dissolution solution adjusted to pH 7.4 using a sodium phosphate buffer (Na₃PO₄ buffer), Compound I was dissolved from substantially all capsules. In addition, Compound I was dissolved within 1 hour from a plurality of capsules.

In an accelerated stability condition, the dissolution results before and after storage were substantially the same.

### Example 20

Preparation of Capsules to which Enteric Coating is Applied

200 mg of Compound I was blended with magnesium stearate and then filled into HPMC capsules. The filled HPMC capsules were enteric coated with coating ingredients of Table 32 below using a fluid bed. The composition of the capsules according to the present method is shown in Table 33.

TABLE 32

| | Coating Suspension | Coating Dry Mixture |
|---|---|---|
| Ingredient | Weight Ratio (%) | Weight Ratio (%) |
| Eudragit L100/S100 (1:1) | 9.1 | 69 |
| Triethyl Citrate | 0.7 | 5 |
| Talc | 3.4 | 26 |
| Anhydrous Ethanol | 86.8 | — |
| Sum | 100 | 100 |

TABLE 33

| Ingredient | Weight Ratio (%) | mg/unit |
|---|---|---|
| Compound I | 99 | 200 |
| Magnesium Stearate | 1 | 2 |
| Sum | 100 | 202 |
| HPMC Capsule | — | 62* |
| Coating Ingredients of Table 32 | — | 84 |

| | |
|---|---|
| Capsule Weight | 348 |

* Average weight of 10 empty capsules

For the enteric coated capsules (Example 20), the dissolution test was performed, and the results are shown in Table 34 below.

TABLE 34

| Dissolution | | |
|---|---|---|
| Environment | Hour | Dissolution Rate of Example 20 (%) |
| Acidic Environment | 2 | No change in capsule |
| (500 ml 0.1 N HCl) | | |
| After Dissolving for 2 | 1 | 44 |
| Hours in Acidic | 2 | 102 |
| Environment, Neutral | 4 | 102 |
| Environment | 6 | 102 |
| (Na₃PO₄, pH 7.4) | | |

As shown in Table 34 above, it was confirmed that when the enteric capsules were exposed to a 0.1 N HCl dissolution solution for 2 hours, there was no change in all the capsules, whereas Compound I was dissolved in a neutral environment.

### Examples 21 to 23

Preparation of Capsules to which Enteric Coating is Applied

Compound I was mixed with Eudragit S100 and Pharmacoat Hypromellose 606 (HPMC). This mixture was subjected to fluid bed granulation processing with top spray nozzles using anhydrous ethanol as a granulating solvent (granulating liquid) to prepare the granules. The resulting granules were dried, then mixed with magnesium stearate, and filled into HPMC capsules. The composition of the capsules according to the present method is shown in Table 35.

TABLE 35

| | | mg/unit | | |
|---|---|---|---|---|
| Ingredient | Weight ratio (%) | Example 21 | Example 22 | Example 23 |
| Compound I | 75.0 | 25.0 | 100.0 | 200.0 |
| Eudragit S100 | 20.0 | 6.67 | 26.7 | 53.3 |
| HPMC | 4.0 | 1.33 | 5.33 | 10.7 |
| (Hypromellose 606) | | | | |
| Magnesium Stearate | 1.0 | 0.33 | 1.33 | 2.67 |
| Anhydrous Ethanol | Removed during process | | | |
| HPMC Capsule | — | 1 Unit | | |
| Sum (Excluding Capsule | 100 | 33.3 | 133.3 | 266.7 |
| Weight) | | | | |

The enteric coated capsules (Examples 21 to 23) were tested for the stability and dissolution. The results are shown in Tables 36 and 37.

TABLE 36
Results of Stability and Dissolution in Long-Term Storage Stability Condition (25°C./
60% RH)

| | | Example 21 | | Example 22 | | Example 23 | |
|---|---|---|---|---|---|---|---|
| | | Storage Period | | | | | |
| | | (25° C./60% RH) | | | | | |
| | | | T = 6 | | T = 6 | | T = 6 |
| | | T = 0 | months | T = 0 | months | T = 0 | months |
| Individual Impurities | | RRT 0.86: | RRT 0.86: | RRT 0.86: | RRT 0.86: | RRT 0.86: | RRT 0.86: |
| (RRT) | | 0.09 | 0.09 | 0.10 | 0.09 | 0.09 | 0.09 |
| | | RRT 0.97: | RRT 0.97: | RRT 0.97: | RRT 0.97: | RRT 0.97: | RRT 0.97: |
| | | 0 | <0.05 | 0 | <0.05 | 0 | <0.05 |
| | | RRT 1.08: | RRT 1.08: | RRT 1.08: | RRT 1.08: | RRT 1.08: | RRT 1.08: |
| | | 0.12 | 0.11 | 0.12 | 0.12 | 0.12 | 0.12 |
| | | RRT 1.17: | RRT 1.17: | RRT 1.17: | RRT 1.17: | RRT 1.17: | RRT 1.17: |
| | | 0.17 | 0.14 | 0.17 | 0.15 | 0.16 | 0.14 |
| Total Impurities (%) | | 0.38 | 0.34 | 0.39 | 0.36 | 0.37 | 0.35 |
| Dissolution | | | | | | | |
| Environment | Hour | | | | | | |
| Acidic | 2 | 0 | 0 | 0 | 0 | 0 | 0 |
| Environment | | | | | | | |
| (500 ml 0.1 N | | | | | | | |
| HCl) | | | | | | | |
| Na₃PO₄•12H₂O | 2.5 | 1 | 0 | 0 | 0 | 0 | 0 |
| Buffer pH | 3.0 | 1 | 0 | 0 | 0 | 0 | 0 |
| 6.0 | | | | | | | |
| Na₃PO₄•12H₂O | 3.5 | 90 | 83 | 61 | 54 | 47 | 45 |
| Buffer pH | 4.0 | 93 | 96 | 95 | 90 | 96 | 91 |
| 7.4 | 5.0 | NA | 97 | NA | 99 | NA | 98 |

TABLE 37
Results of Stability and Dissolution in Accelerated Stability Condition
(40° C./75% RH)

| | | Example 21 | | Example 22 | | Example 23 | |
|---|---|---|---|---|---|---|---|
| | | Storage Period | | | | | |
| | | (40° C./75% RH) | | | | | |
| | | | T = 6 | | T = 6 | | T = 6 |
| | | T = 0 | months | T = 0 | months | T = 0 | months |
| Individual Impurities | | RRT 0.86: | RRT 0.86: | RRT 0.86: | RRT 0.86: | RRT 0.86: | RRT 0.86: |
| (RRT) | | 0.09 | 0.08 | 0.10 | 0.08 | 0.09 | 0.08 |
| | | RRT 1.08: | RRT 1.08: | RRT 0.97: | RRT 0.97: | RRT 0.97: | RRT 0.97: |
| | | 0.12 | 0.14 | 0 | <0.05 | 0 | 0.05 |
| | | RRT 1.17: | RRT 1.17: | RRT 1.08: | RRT 1.08: | RRT 1.08: | RRT 1.08: |
| | | 0.17 | 0.16 | 0.12 | 0.12 | 0.12 | 0.12 |
| | | | | RRT 1.17: | RRT 1.17: | RRT 1.17: | RRT 1.17: |
| | | | | 0.17 | 0.15 | 0.16 | 0.16 |
| Total Impurities (%) | | 0.38 | 0.38 | 0.39 | 0.35 | 0.37 | 0.41 |
| Dissolution | | | | | | | |
| Environment | Hour | | | | | | |
| Acidic | 2 | 0 | 0 | 0 | 0 | 0 | 0 |
| Environment | | | | | | | |
| (500 ml 0.1 | | | | | | | |
| N HCl) | | | | | | | |
| Na3PO4•12H2O | 2.5 | 1 | 0 | 0 | 0 | 0 | 1 |
| Buffer pH | 3.0 | 1 | 1 | 0 | 0 | 0 | 2 |
| 6.0 | | | | | | | |
| Na3PO4•12H2O | 3.5 | 90 | 77 | 61 | 65 | 47 | 48 |
| Buffer pH | 4.0 | 93 | 93 | 95 | 97 | 96 | 90 |
| 7.4 | 5.0 | NA | 95 | NA | 99 | NA | 99 |

As shown in Tables 36 and 37 above, it was confirmed that Compound I was stable for 6 months in a long-term storage stability condition (25° C./60% RH) and an accelerated stability condition (40° C./75% RH), and there was no generation of impurities or increase in impurities.

As a result of the dissolution test, it was confirmed that Compound I was not substantially dissolved in an acidic environment, and Compound I was dissolved in a neutral environment, and the dissolution results before and after storage were substantially the same.

Eventually, the enteric coated capsule of the present invention can delay the dissolution of Compound I until it reaches a non-acidic environment in which Compound I can be rapidly released, and thus the enteric coated capsule of the present invention is very useful as a dosage form of therapeutic agents for inflammatory bowel diseases, which require the release of the active ingredient such as Compound I into lesions of the lower small intestine or the large intestine.

1. A pharmaceutical formulation comprising a sodium palmitoyl-L-prolyl-L-prolyl-glycyl-L-tyrosinate salt of Formula 1 below.

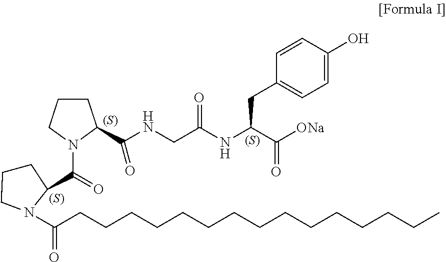

2. The pharmaceutical formulation for oral administration according to claim 1, characterized in that the pharmaceutical formulation is in the form of a tablet or capsule.

3. A pharmaceutical formulation for oral administration comprising a sodium palmitoyl-L-prolyl-L-prolyl-glycyl-L-tyrosinate salt of Formula 1 below and an enteric polymer.

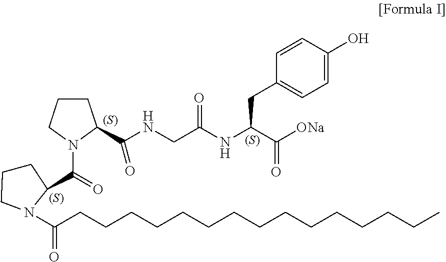

4. The pharmaceutical formulation according to claim 3, characterized in that the enteric polymer is at least one selected from the group consisting of a methacrylic acid-methyl methacrylate copolymer, a methyl acrylate-methyl methacrylate-methacrylic acid copolymer, a methacrylic acid-ethyl acrylate copolymer, cellulose acetate phthalate, hydroxypropyl methylcellulose phthalate, hydroxypropyl methylcellulose acetate succinate, polyvinyl acetate phthalate, cellulose acetate trimellitate, carboxymethyl ethyl cellulose, and shellac.

5. The pharmaceutical formulation according to claim 3, characterized in that the enteric polymer is a methacrylic acid-methyl methacrylate copolymer, a methyl acrylate-methyl methacrylate-methacrylic acid copolymer, or a mixture thereof.

6. The pharmaceutical formulation according to claim 3, characterized in that the enteric polymer is a methacrylic acid-methyl methacrylate 1:1 copolymer, a methacrylic acid-methyl methacrylate 1:2 copolymer, or a mixture thereof.

7. The pharmaceutical formulation according to claim 3, characterized in that the enteric polymer is a methacrylic acid-methyl methacrylate 1:2 copolymer.

8. The pharmaceutical formulation according to claim 3, characterized in that the enteric polymer comprises a methacrylic acid-methyl methacrylate 1:1 copolymer and a methacrylic acid-methyl methacrylate 1:2 copolymer in a weight ratio of 1:1.

9. The pharmaceutical formulation according to claim 3, characterized in that the pharmaceutical formulation comprises the enteric polymer in an amount of 10 to 300 parts by weight based on 100 parts by weight of the sodium palmitoyl-L-prolyl-L-prolyl-glycyl-L-tyrosinate salt.

10. The pharmaceutical formulation according to claim 3, characterized in that the pharmaceutical formulation comprises the enteric polymer in an amount of 20 to 80 parts by weight based on 100 parts by weight of the sodium palmitoyl-L-prolyl-L-prolyl-glycyl-L-tyrosinate.

11. The pharmaceutical formulation according to claim 3, characterized in that the pharmaceutical formulation further comprises at least one additive selected from the group consisting of microcrystalline cellulose, mannitol, hydroxypropyl methylcellulose (HPMC), polyethylene oxide, sodium croscarmellose, crospovidone, polyoxyglyceride, magnesium aluminometasilicate, magnesium stearate, talc, and sodium starch glycolate.

12. The pharmaceutical formulation according to claim 3, characterized in that the pharmaceutical formulation further comprises at least one additive selected from the group consisting of magnesium stearate, sodium starch glycolate, talc, and triethyl citrate (TEC).

13. The pharmaceutical formulation according to claim 3, characterized in that the pharmaceutical formulation comprises a methacrylic acid-methyl methacrylate 1:2 copolymer as the enteric polymer, and further comprises hydroxypropyl methylcellulose (HPMC) and magnesium stearate.

14. The pharmaceutical formulation according to claim 3, characterized in that sodium palmitoyl-L-prolyl-L-prolyl-glycyl-L-tyrosinate is dissolved at pH 6.0 or higher.

15. The pharmaceutical formulation according to claim 3, characterized in that in the dissolution test at 37° C. and 100 rpm according to the United States Pharmacopeia (USP) type 2 paddle method, 20% or less of sodium palmitoyl-L-prolyl-L-prolyl-glycyl-L-tyrosinate is dissolved in a pH 6.0 buffer for 1 hour, and 80% or more of sodium palmitoyl-L-prolyl-L-prolyl-glycyl-L-tyrosinate is dissolved in a pH 7.4 buffer for 1 hour.
